(19) United States
(12) Patent Application Publication
(10) Pub. No.: US 2016/0038311 A1
(43) Pub. Date: Feb. 11, 2016

(54) LAYERING TECHNIQUE FOR AN ADJUSTABLE, REPAIRABLE VARIABLE STIFFNESS PROSTHETIC FOOT

(71) Applicant: Board of Regents, The University of Texas System, Austin, TX (US)

(72) Inventors: Roger V. Gonzalez, El Paso, TX (US); Joshua Bowen, La Habra, CA (US); Aaron Nystrom, El Paso, TX (US)

(73) Assignee: Board of Regents, The University of Texas System

(21) Appl. No.: 14/819,059

(22) Filed: Aug. 5, 2015

Related U.S. Application Data
(60) Provisional application No. 62/034,992, filed on Aug. 8, 2014.

(51) Int. Cl. A61F 2/50; A61F 2/66

(52) U.S. Cl. CPC A61F 2/5044; A61F 2/6607; A61F 2002/5024; A61F 2002/503; A61F 2002/5056; A61F 2002/5079; A61F 2002/5083; A61F 2002/607

(57) ABSTRACT

A modular prosthetic foot includes a keel section and in some cases, an optional heel section. The keel section may also include an optional toe section. The keel section can include separate layers of various stiffnesses, wherein the optional toe section and the optional heel section are composed of varying stiffness layers nested together. The stiffness of the keel section is adjustable by a user without specialized tools. The components of the prosthetic foot are modular and can be removed and exchanged without altering the fit and alignment of the prosthetic foot.

### CROSS-REFERENCE TO PROVISIONAL APPLICATION

This application claims priority under 35 U.S.C. 119(e) to U.S. Provisional Patent Application Ser. No. 62/034,992, entitled “Layering Technique for an Adjustable, Repairable Variable Stiffness Prosthetic Foot,” which was filed on Aug. 8, 2014, the disclosure of which is incorporated herein by reference in its entirety.

### TECHNICAL FIELD

Embodiments are related to an external prosthesis and, more particularly, to a prosthetic foot wherein the keel stiffness thereof may be adjusted and customized to match the weight, gait, and activity level of the wearer utilizing the prosthetic foot.

### BACKGROUND OF THE INVENTION

People living in developing and underdeveloped countries often have limited or no access to prosthetic limbs. Current prosthetic limbs are usually too expensive for people in those areas of the world to afford. Prosthetic feet tend to be expensive in part because they are complex structures that are custom manufactured in small quantities to meet specific user needs. There is, therefore, a need for an affordable prosthetic foot that provides customizable comfort and stability.

A prosthetic foot is usually characterized by a low profile, elongated forefoot portion incorporating an attachment section, a keel section, a toe section, and a heel. Each of the available low-cost solutions has significant deficiencies. Specifically, the component parts of the prosthesis are often permanent, not repairable, not customizable, and too heavy or rigid. As a result, these other low-cost prosthetics do not have an appropriate dynamic response characteristic of the human foot.

A prosthetic foot can be constructed with a standard stiffness that may not accommodate the needs of all users. Other prosthetic feet may provide variable stiffness, but require the manufacturer of the foot to adjust the stiffness. Normal practice in the industry is to replace an uncomfortable or ill-fitting foot with a completely new one. Therefore, a need exists for an improved prosthetic foot with easily varied and customized stiffness.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to relate to improved prosthetic limbs with layers that offer variable stiffness.

It is another aspect of the disclosed embodiments to provide a customizable prosthetic foot with modular components that can be removed and exchanged without altering the fit and alignment of the entire prosthesis.

It is a further aspect of the disclosed embodiments to provide an external prosthetic foot with variable keel stiffness.

In one embodiment, a prosthetic foot can be implemented which includes a group of removable layers that offer variable stiffness. The foot can be composed of n layers, wherein such layers are composed of a first set of plies oriented at 45°, a second set of plies oriented at 0°, and a third set of plies oriented at 45°. Layer stiffness increases as the number of 0° plies in the second set is increased. Layers of different stiffness can be combined such that the stiffness of the prosthetic foot is variable and customized according to user needs.

In another embodiment, a prosthetic foot can be implemented with a group of layers that offer variable stiffness, wherein such layers can include a first layer, a second layer, and a third layer. The first layer can include at least one of a soft layer, a medium layer, and a hard layer; and the second layer can include at least one of a soft layer, a medium layer, and a hard layer. The third layer can be composed of at least one of a soft layer, a medium layer, and a hard layer. Again, the keel stiffness of the prosthetic foot is variable and customized according to user needs.

In yet another embodiment, a modular prosthetic foot can be implemented, which includes a keel section. The section may include an optional heel section. The keel section may also include an optional toe section. The keel section can include separate layers of varying stiffness, wherein the optional toe section and the optional heel section are composed of varying stiffness layers nested together. The stiffness of the keel section is adjustable by a user without specialized tools. The components of the prosthetic foot are modular and can be removed and exchanged without altering the fit and alignment of the prosthetic foot.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates an exemplary pictorial illustration of the prosthetic foot with adjustable layers for variable stiffness, in accordance with a preferred embodiment;

FIG. 2 illustrates an exemplary pictorial illustration of the prosthetic foot with a pyramid adapter, ankle, toe layer, and heel layer, in accordance with the an alternative embodiment;

FIG. 3 illustrates an exemplary pictorial illustration of the prosthetic foot with a pyramid adapter, ankle block, toe layer, and heel layer, in accordance with an alternative embodiment; and

FIG. 4 illustrates an exemplary graphical illustration of force deflection curves of the individual stiffness layers and combinations of stiffness layers, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

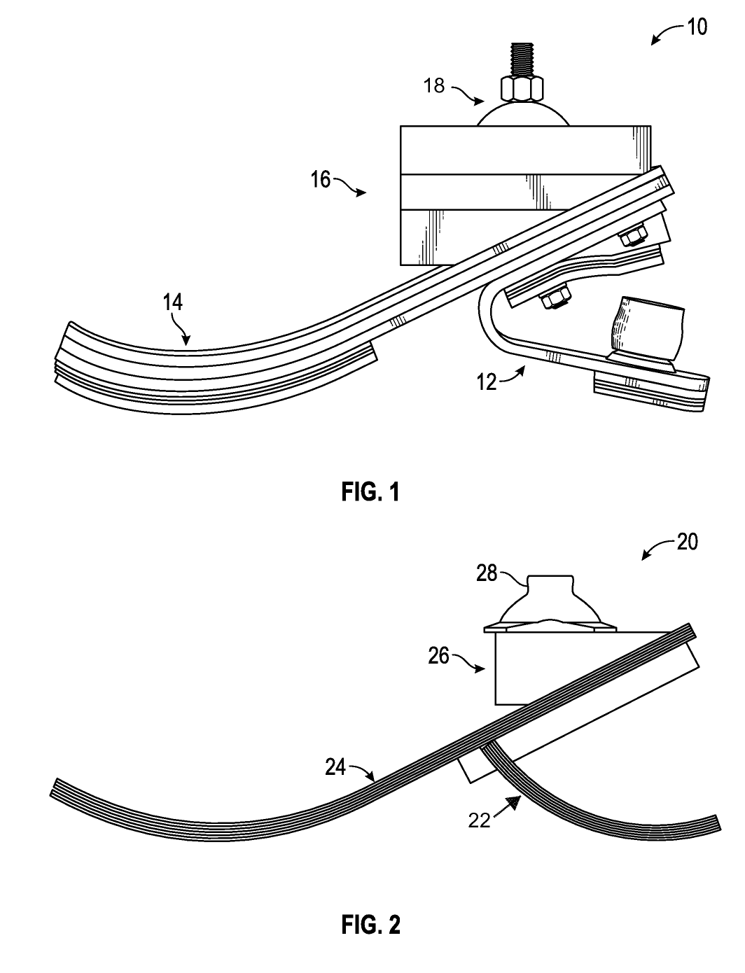

FIG. 1 illustrates an exemplary pictorial illustration of a prosthetic foot 10 with adjustable layers for variable stiffness, in accordance with a preferred embodiment. The prosthetic foot 10 includes one or more toe layers 14 that connect to one or more heel layers 12. An ankle block 16 is located above and toward the rear of the toe layers 14. A pyramid adapter 18 connects to and sits above the ankle block 16.

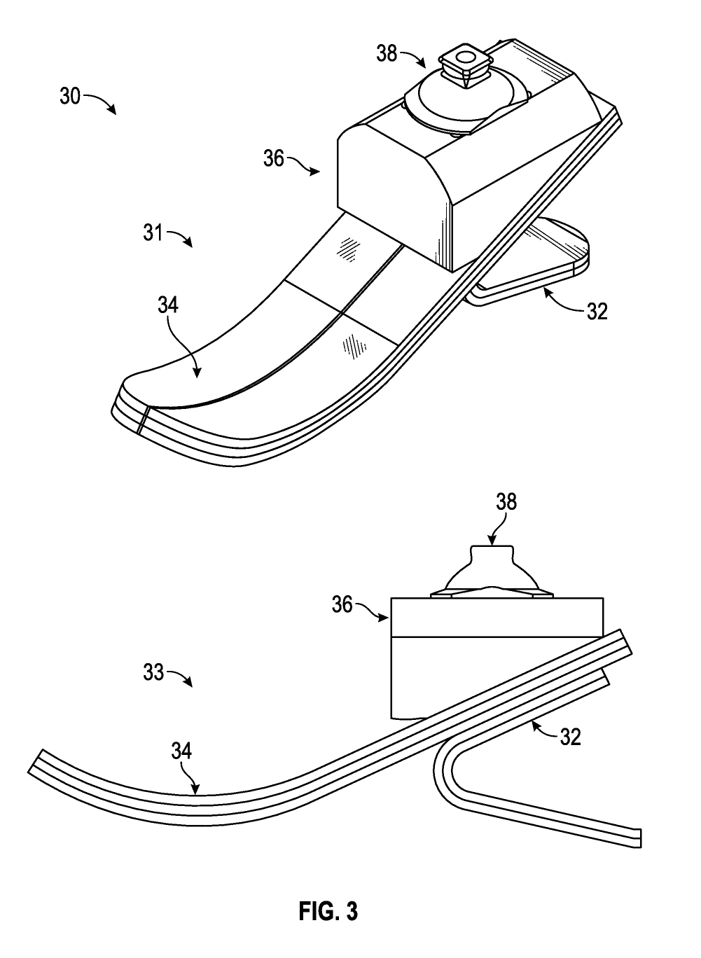

The configuration depicted in FIG. 1 (and also depicted in FIGS. 2-3) illustrates a modular, external prosthetic foot with a keel assembled from multiple layers of various stiffnesses. The stiffness of each layer can vary from one layer to another. Assembling the keel from layers with varying stiffnesses allows the keel stiffness to be adjusted and customized. The toe and heel sections or layers of the keel of the prosthetic foot are assembled by nesting several layers together, as illustrated in FIG. 1.

FIG. 2 illustrates an exemplary pictorial illustration of a prosthetic foot 20 with a pyramid adapter 28, an ankle block 26, a toe layer 24, and a heel layer 22, in accordance with an alternative embodiment. Removable layers can be attached to the prosthetic foot 20 without adjusting or removing parts of the prosthetic foot that attach it to the remaining prosthesis, thus allowing stiffness layers to be added, removed, or exchanged.

FIG. 3 illustrates an exemplary pictorial illustration of a prosthetic foot 30 with a pyramid adapter 38, an ankle block 36, a heel layer 32 and a toe layer 34, in accordance with an alternative embodiment. In the illustration of FIG. 3, a side view 33 of the prosthetic foot 30 is shown along with an angular perspective view 31 of the prosthetic 30. The modular prosthetic feet shown in FIGS. 1-3 can be customized to the users' weight, height, and activity level. Each layer can be exchanged to vary the stiffness of the prosthetic foot according to the needs of a user. This allows the prosthetic foot to be customized to the patient being fit with prosthetic foot. Thus, for example, the toe layers 34 and the heel layers 32 can be added, removed, or exchanged.

The disclosed embodiments (preferred and/or alternative) improve upon the modular prosthetic foot by allowing the foot to be repaired and adjusted without removing the prosthetic foot from the remainder of the prosthesis. The stiffness of the prosthetic foot can be adjusted to the patient by a clinician without the need to purchase a new prosthetic foot or send it back to the manufacturer. The disclosed embodiments allow prosthetic feet to be customized immediately by clinicians at the office and without the need to order a new prosthetic foot. Also, amputees can be provided with replacement parts and can repair the foot without the need to return to the clinic. The components of the disclosed prosthetic feet 10, 20, and 30 shown respectively at FIGS. 1, 2, and 3 are modular and can be removed and exchanged without altering the fit and alignment of the entire prosthesis.

Layer stiffness depends on ply (plies) orientation and the number of plies within the layer. For example, any given layer can be constructed to be soft, medium, and/or hard. A plurality of layers, preferably three layers as illustrated in FIGS. 1 and 3 can be nested together in various combinations to create the keel of the prosthetic foot with varying stiffness. A user can request three layers comprising: soft-soft-soft; medium-medium-medium; hard-hard-hard; soft-soft-medium; soft-medium-medium, etc. The stiffness of the foot varies according to combination of layers selected for the user. By changing the stiffness of the layers, the prosthetic foot is customized to the user. It is understood that the structural components are not necessarily comprised of composite materials and can be constructed of a variety of materials.

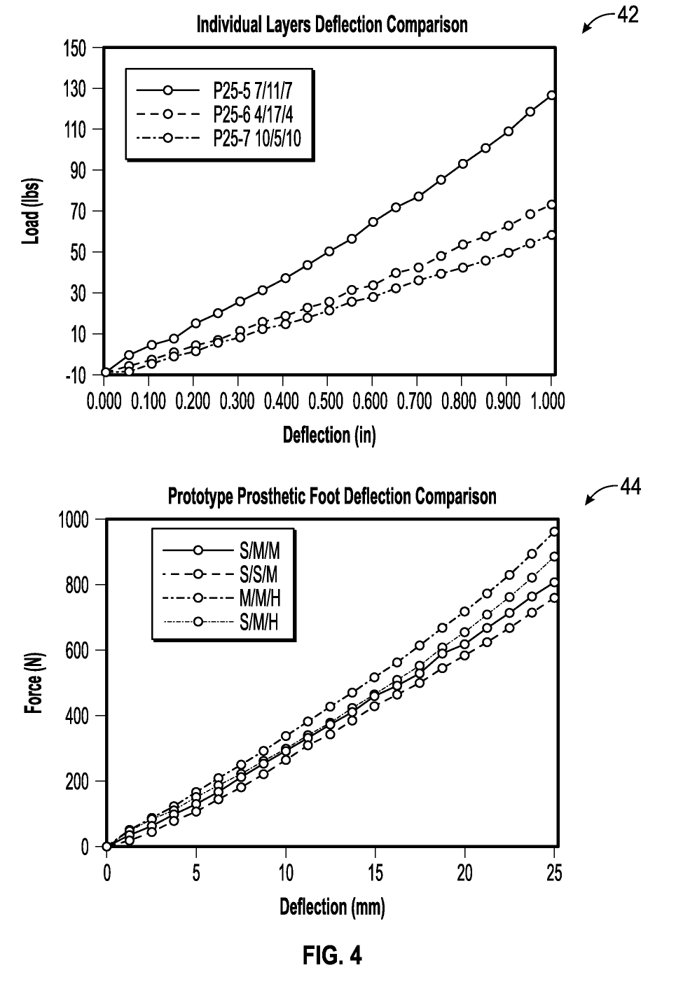

FIG. 4 illustrates graphics 42 and 44, which respectively depict sample force deflection curves of the disclosed prosthetic foot, in accordance with example embodiments. Graph 42 illustrates data indicative of the comparison of the deflection of individual layers. Graph 44 depicts data indicative of the comparison of prototype prosthetic foot deflection. It can be appreciated that the data shown in graphs 42, 44 of FIG. 4 is exemplary only and is not considered a limiting feature of the disclosed embodiments.

The following example of ply layers and orientations is also exemplary and does not limit the disclosed embodiments solely to such ply layers and orientations. The Individual Layers Deflection Comparison graph 42 shown in FIG. 4 illustrates the force deflection of each layer that could be used to construct the keel of the prosthetic foot. P25-7 represents the Soft Layer; P25-5 represents the Medium Layer; and P25-6 represents the Hard Layer. P25-5—7/11/7 is Layer “P25-5” (the layer is 25 plies thick and the 5th prototype); 7/11/7 is the composition of the composite plies within the layer: 7 plies oriented at 45°; 11 plies oriented at 0°; and 7 plies oriented at 45°. It can be appreciated that such parameters and data are exemplary only and do not constitute limiting features of the disclosed embodiments.

Such data indicates how layering can be configured with the composite so that the layer(s) can be remade and layered in the same manner. Everything is symmetrical regarding ply layering so the “11” plies at 0° are in the middle of the layer and sandwiched by 7 plies at 45° for a total of 25 plies to make up that layer. P25-6—4/17/4 similarly has 4 plies at 45°, 17 at 0°, and 4 at 45°. P25-7—10/5/10 has 10 plies at 45°; 5 plies at 0°: and 10 plies at 45°. The more 0° plies in the layer the stiffer the layer. It is understood that soft, medium, and hard layers are exemplary layers in the preferred embodiment. It can be appreciated that the disclosed embodiments can include additional layers such as soft, super soft, medium/hard, medium soft, hard/very hard, etc.

A Prototype Prosthetic Foot Deflection Comparison graph 44 shown in FIG. 4 indicates the force deflection of the prosthetic when assembled with various combinations of stiffness layers (i.e., soft, medium, hard). Exemplary combinations shown in the graph include: soft/medium/medium; soft/soft/medium; medium/medium/hard; and soft/medium/hard.

Based on the foregoing, it can be appreciated that a number of embodiments, preferred and alternative, are disclosed herein. For example, in one embodiment, a prosthetic foot can be implemented which includes a group of removable layers that offer variable stiffness. The foot can be composed of n layers, wherein such layers are composed of a first set of plies oriented at 45°, a second set of plies oriented at 0°, and a third set of plies oriented at 45°. Layer stiffness increases as the number of 0° plies in the second set is increased. Layers of different stiffnesses can be combined such that the stiffness of the prosthetic foot is variable and customized according to user needs.

In another embodiment, a prosthetic foot can be implemented with a group of layers that offer variable stiffness, wherein such layers can include a first layer, a second layer, and a third layer. The first layer can include at least one of a soft layer, a medium layer, and a hard layer; and the second layer can include at least one of a soft layer, a medium layer, and a hard layer. The third layer can be composed of at least one of a soft layer, a medium layer, and a hard layer. Again, the keel stiffness of the prosthetic foot is variable and customized according to user needs.

In yet another embodiment, a modular prosthetic foot can be implemented, which includes a keel section. The section may include an optional heel section. The keel section may also include an optional toe section. The keel section can include separate layers of varying stiffness, wherein the optional toe section and the optional heel section are composed of varying stiffness layers nested together. The stiffness of the keel section is adjustable by a user without specialized tools. The components of the prosthetic foot are modular and can be removed and exchanged without altering the fit and alignment of the prosthetic foot.

It will be appreciated that variations of the above-disclosed and other features and functions, or alternatives thereof, may be desirably combined into many other different systems or applications. Furthermore, it can be appreciated that various presently unforeseen or unanticipated alternatives, modifications, variations or improvements therein may be subsequently made by those skilled in the art which are also intended to be encompassed by the following claims.

What is claimed is:

1. A prosthetic foot, comprising:
a plurality of removable layers of various stiffnesses, wherein said plurality of removable layers of various stiffnesses comprises n layers;
wherein said plurality of removable layers of various stiffnesses is composed of a first set of plies, a second set of plies, and a third set of plies;
wherein a stiffness of said prosthetic foot increases with an increased number of said second set plies in a layer; and
wherein a keel stiffness of said prosthetic foot is variable and customized according to the needs of a user of said prosthetic foot.

2. The prosthetic foot of claim 1 wherein said plurality of removable layers of various stiffnesses contains said first set of plies oriented at 45°.

3. The prosthetic foot of claim 1 wherein said plurality of removable layers of various stiffnesses contains said second set of plies oriented at 0°.

4. The prosthetic foot of claim 1 wherein said plurality of removable layers of various stiffnesses contains said third set of plies oriented at 45°.

5. The prosthetic foot of claim 1 wherein said stiffness increases of said prosthetic foot with an increased number of said second set of plies oriented at 0° in a layer.

6. The prosthetic foot of claim 1 wherein said plurality of removable layers of various stiffnesses is composed of said first set of plies oriented at 45°, said second set of plies oriented at 0°, and said third set of plies oriented at 45°.

7. The prosthetic foot of claim 6 wherein said stiffness of said prosthetic foot increases with an increased number of said second set plies oriented at 0° in a layer.

8. A prosthetic foot, comprising:
a plurality of layers of various stiffnesses, wherein said plurality of layers of various stiffnesses comprises a first layer, a second layer, and a third layer;
said first layer comprising at least one of a soft layer, a medium layer, and a hard layer;
said second layer comprising at least one of a soft layer, a medium layer, and a hard layer;
said third layer comprising at least one of a soft layer, a medium layer, and a hard layer; and
wherein a keel stiffness of said prosthetic foot is variable and customized according to the needs of a user.

9. The prosthetic foot of claim 1 further comprising an ankle block.

10. The prosthetic foot of claim 1 further comprising a pyramid adaptor.

11. The prosthetic foot of claim 8 wherein at least one of said first, second or third layers comprises a toe layer.

12. The prosthetic foot of claim 8 wherein at least one of said first, second or third layers comprises a heel layer.

13. The prosthetic foot of claim 8 further comprising an ankle block and a pyramid block.

14. The prosthetic foot of claim 13 wherein at least one of said first, second or third layers comprises a toe layer.

15. The prosthetic foot of claim 13 wherein at least one of said first, second or third layers comprises a heel layer.

16. A modular prosthetic foot comprising:
a keel section, wherein said keel section may comprise an optional heel section; wherein said keel section may comprise an optional toe section;
said keel section comprising separate layers of varying stiffness;
wherein a stiffness of said keel section is adjustable without specialized tools; and
wherein components of said prosthetic foot are modular and removable and exchangeable without altering a fit and an alignment of said prosthetic foot.

17. The prosthetic foot of claim 16 wherein said optional toe section comprises a plurality of layers of various stiffnesses nested together.

18. The prosthetic foot of claim 16 wherein said optional keel section comprises a plurality of layers of various stiffnesses nested together.

19. The prosthetic foot of claim 17 wherein said optional keel section comprises a plurality of layers of various stiffnesses nested together.

20. The prosthetic foot of claim 16 wherein said optional toe section and said optional keel section each comprise a plurality of layers of various stiffnesses nested together.
